(19) United States
(12) Patent Application Publication
(10) Pub. No.: US 2018/0193507 A1
(43) Pub. Date: Jul. 12, 2018

(54) Apparatus for Ozone Gas Disinfection of Closed Conduits

(71) Applicant: ClearWater Tech, LLC, San Luis Obispo, CA (US)

(72) Inventors: Cameron Tapp, San Luis Obispo, CA (US); Christopher R. Schulz, Aurora, CO (US); Philip J. Steckling, San Luis Obispo, CA (US)

(21) Appl. No.: 15/868,770

(22) Filed: Jan. 11, 2018

Related U.S. Application Data
(60) Provisional application No. 62/445,102, filed on Jan. 11, 2017.

(51) Int. Cl. A61L 2/20; C02F 1/00; B08B 9/032

(52) U.S. Cl. CPC A61L 2/202; C02F 1/008; B08B 9/0328; C02F 2209/235; C02F 2209/003; C02F 2303/04; C02F 2201/782

(57) ABSTRACT

Methods and systems for disinfecting conduits using gaseous ozone are disclosed. The systems and methods include an ozone generation apparatus capable of producing gaseous ozone to be introduced into conduits. The gaseous ozone can then disinfect the conduit. Some embodiments of the systems and methods further comprise an ozone destruction apparatus capable of destroying the ozone after a certain stage of the disinfection process is reached.

### CROSS-REFERENCES TO RELATED APPLICATIONS

The present application claims priority to U.S. Provisional Patent Application No. 62/445,102, filed Jan. 11, 2017, entitled “Apparatus for Ozone Gas Disinfection of Closed Conduits,” which is hereby incorporated by reference in its entirety.

### STATEMENT REGARDING FEDERALLY SPONSORED RESEARCH

Not Applicable.

### BACKGROUND OF THE INVENTION

### 1. Field of the Invention

This invention relates to an apparatus and method for disinfecting a fluid carrying conduit. More particularly, this invention relates to a method and apparatus for quickly disinfecting water pipelines and other conduits through the introduction of gaseous ozone into the pipelines and conduits.

### 2. Description of the Related Art

Microbial contamination within new or repaired water mains has been associated with several waterborne disease outbreaks in public water supply systems. Currently, chlorine is the most commonly-utilized disinfectant for treating water mains and conduits. Practices recommended by the American Water Works Association to treat the interior of water-carrying conduits include several techniques that have a number of shortcomings, including the handling and on-site preparation of hazardous chemical solutions, uncertainty of the effectiveness of the treatment, the need to carry out a dechlorination step before disposal of the chlorinated discharges, the need to dispose of large volumes of dechlorinated water, and the length of exposure time of the chlorine in the pipeline that is necessary to ensure adequate disinfection.

Among the treatment methods currently utilized are the continuous feed method, the slug method, and the tablet method. In the continuous feed method, the conduit is first flushed with a strong chlorine solution, then filled with a solution having at least 25 mg/L of free chlorine. That solution is retained within the conduit so that a residual of at least 10 mg/L is maintained after the passage of 24 hours. In the slug method, a slug dose of free chlorine having a concentration greater than 100 mg/L is moved slowly through the conduit so that all interior surfaces are exposed to the highly concentrated chlorine solution for a period of not less than three hours. Finally, in the tablet method, calcium hypochlorite tablets are attached to the conduit inner surface at several axially spaced positions. Then the conduit is filled with water to dissolve the tablets, such that a residual of at least 25 mg/L is maintained in contact with the conduit inner surface for at least 24 hours.

Although generally effective, the methods presently employed have several drawbacks. The slug and continuous feed method require the use, transport, and on-site preparation of hazardous hypochlorite and sodium bisulfate solutions in trailer or truck-mounted storage tanks for the chlorination and dechlorination steps. The 24-hour minimum holding time for the slug and continuous feed methods to ensure adequate disinfection involves lengthy delays that adversely affect construction time schedules. Sometimes the tablets used in the tablet method do not fully dissolve within the conduit, resulting in improper disinfection. Because the water in the tablet method is static, incomplete dissolving of the tablets can result in local areas of ineffective disinfection. Finally, all the aforementioned chlorine-based methods requires dechlorination of the treatment solutions to allow disposal by discharge of the solutions into sanitary or storm sewers, storage ponds, or flood control channels.

In addition to the material handling, disposal, and time delay factors noted above, the conduit disinfection methods in common use today are also not linked to a scientifically rational disinfection basis. The concentration and exposure time criteria are relatively arbitrary, as contrasted with the disinfectant concentration times contact time (CT) concepts that form the basis for disinfection in modern drinking water treatment systems.

Instead of using chlorine, others have contemplated the use of ozone for disinfecting newly constructed or rehabilitated water mains. A “flow through disinfection method” has been used, where a water main is filled with finished water. An ozone solution is then injected into the water supply, at a rate such that an outlet ozone residual of 0.1 to 0.2 mg/L is maintained for a certain exposure time in the water main. The ozonated water is continuously fed under pressure into the water main until the disinfection parameters (ozone residual and exposure time) are met, and then an operator drains and flushes the main with water from the distribution system before the main is returned to service. While this method proved to be effective for disinfecting water mains of smaller diameter and length, the decaying nature of ozone in water restricted the application of ozone disinfection to these smaller scale operations. The method of injecting ozonated water into a main typically does not maintain adequate concentrations to disinfect water mains of larger length and diameter.

Therefore, a need in the art exists for a faster, more reliable method of disinfecting water mains and other conduits.

### SUMMARY OF THE INVENTION

The present invention provides a system for disinfecting pipes. The system comprises a conduit and an ozone generation apparatus for producing gaseous ozone. The conduit has an inlet, an outlet, and an interior volume which is in fluid communication with the inlet and the outlet. The ozone generation apparatus is in fluid communication with the conduit inlet, and the interior volume of the conduit is substantially free of liquid. The ozone generation apparatus may also comprise a blower or air compressor for moving fluid from the inlet, through the interior volume, and to the conduit outlet. The ozone generation apparatus may also comprise an ozone analyzer. In some embodiments of the invention, the system further comprises an ozone destruction apparatus in fluid communication with the conduit outlet, which may also comprise an ozone analyzer.

In another embodiment of the invention, a system for disinfecting pipes comprises a conduit, an ozone generation apparatus, and an ozone destruction apparatus. The conduit has an inlet, an outlet, and an interior volume which is in fluid communication with the inlet and the outlet. The ozone generation apparatus is in fluid communication with the conduit inlet, while the ozone destruction apparatus is in fluid communication with the conduit outlet. In some embodiments of the invention, the system further comprises at least one ozone analyzer. The ozone analyzer may be located on the ozone generation apparatus, ozone destruction apparatus, or both, and may be a UV adsorption analyzer. The ozone destruction apparatus may also comprise a catalyst for catalytically converting ozone gas to oxygen gas and releasing the oxygen gas to atmosphere. The catalyst may be one of manganese dioxide, ferric oxide, platinum, combinations thereof, or may be made from other materials.

In some embodiments, the system may further comprise a controller in electrical communication with the ozone generation apparatus and the ozone analyzer. The controller may be configured to execute a program stored in the controller to adjust the rate of gaseous ozone production after a specified concentration of ozone is reached within the interior volume, or an ozone exposure time is reached within the interior volume, or a calculated product of concentration of ozone and ozone exposure time is reached within the interior volume.

Also included within the invention is a process for disinfecting pipes. The process comprises disinfecting a new pipeline prior to introducing liquid into the pipe or removing liquid from an existing conduit, which has an inlet, an outlet, and an interior volume in fluid communication with the inlet and outlet. Ozone is generated with an ozone generation apparatus, and then the generated gaseous ozone is introduced into the interior volume of the conduit. In some embodiments, the process further comprises destroying a portion of the generated ozone at the outlet of the main. The process may also include diluting the gaseous ozone by increasing the volume of gas delivered to the interior volume with an air blower, or blowing a portion of the generated ozone out of the interior volume, or both. Additionally, the process may comprise introducing compressed air into the interior volume of the conduit, with or without gaseous ozone to increase both the volume of gas and the gas pressure.

In another embodiment of the invention, a process for disinfecting pipes comprises removing liquid from a conduit, which has an inlet, an outlet, and an interior volume in fluid communication with the inlet and outlet. Ozone is generated with an ozone generation apparatus, and then the generated ozone is introduced into the interior volume of the conduit. Then, the generated ozone is destroyed at the outlet of the main with oxygen being released to atmosphere. In some embodiments, the ozone concentration within the interior volume of the conduit is monitored. The process further comprises pressurizing the interior volume of the conduit to between 0.1 and 15 psi in some embodiments to promote penetration of gaseous ozone into the pipe wall surfaces. Optionally, the step of destroying a portion of the generated ozone is performed by bringing the ozone into contact with a catalyst.

It is therefore an advantage of the invention to provide a system and process for disinfecting pipes that utilizes gaseous ozone as a disinfectant that improves disinfection efficacy at shorter exposure times than other types of pipeline disinfection methods using liquid ozone, chlorine gas or sodium hypochlorite.

These and other features, aspects, and advantages of the present invention will become better understood upon consideration of the following detailed description, drawings and appended claims.

### BRIEF DESCRIPTION OF THE DRAWINGS

FIG. 1A is a schematic view of an ozone disinfection system according to an embodiment of the present disclosure.

FIG. 1B is a cross-sectional view of the ozone disinfection system of FIG. 1A, taken along line 1B-1B of FIG. 1A.

FIG. 2 is a schematic view of an ozone disinfection system according to another embodiment of the present disclosure.

FIG. 3 is a schematic view of an embodiment of an ozone disinfection system coupled to a pipeline segment, according to an embodiment of the present disclosure.

FIG. 4 is a side view of an ozone generation apparatus that can be present in the ozone disinfection systems of FIGS. 1A-3.

FIG. 5A is a top perspective view of internal components present in the ozone generation apparatus of FIG. 4.

FIG. 5B is another top perspective view of internal components that may be present in the ozone generation apparatus of FIG. 4.

FIG. 6A is a partial view of a control panel that can be used on the ozone generation apparatus of FIG. 4.

FIG. 6B is a partial view of a control panel that can be used on the ozone generation apparatus of FIG. 4.

FIG. 7A is a top perspective view of an ozone destruction apparatus that can be present within the ozone disinfection systems of FIGS. 1A-3.

FIG. 7B is a front view of a control panel that can be used on the ozone destruction apparatus of FIG. 7A.

FIG. 8 is a perspective view of an ozone analyzer that can be present within the ozone disinfection systems of FIGS. 1A-3.

FIG. 9 is a schematic view of a program that could be stored in the memory of a controller in within the ozone disinfection systems of FIGS. 1A-3.

FIG. 10 is a block diagram of an ozone disinfecting process according to an embodiment of the invention.

FIG. 11 is a block diagram of an ozone disinfecting process according to another embodiment of the invention.

FIG. 12 shows an example of a cycle time using the ozone disinfecting process of FIG. 10 or 11.

Like reference numerals will be used to refer to like parts from Figure to Figure in the following description of the drawings.

### DETAILED DESCRIPTION OF THE INVENTION

Before the present systems and methods are described, it is to be understood that the terminology used herein is for the purpose of describing particular embodiments only, and is not intended to limit the scope of the present invention.

It must be noted that as used herein and in the appended claims, the singular forms “a”, “an”, and “the” include plural references unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. It should be understood that the terms “pipe” and “conduit” are interchangeable for purposes of this disclosure.

The present disclosure is directed to systems and processes which can disinfect pipes using gaseous ozone that provide superior disinfection over currently-used methods. The systems and processes reduce treatment time at economical ozone doses to perform proper disinfection. The processes and systems also avoid generating chlorinated water waste streams that would require declorination, disposal, typically in a storm drain or sewer, after the disinfection process is complete.

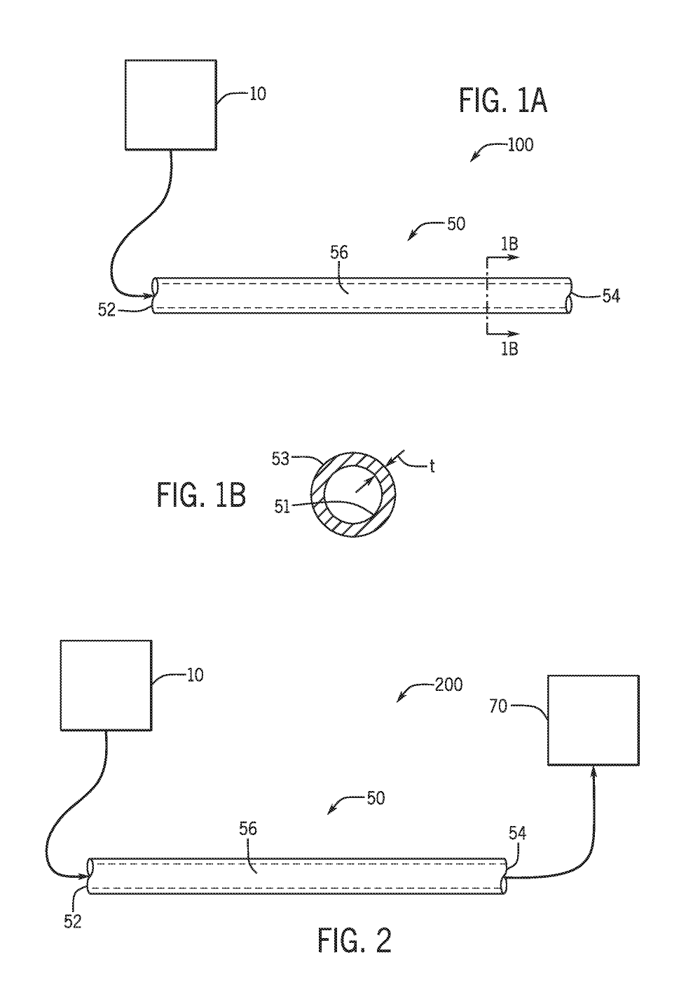

FIGS. 1A-1B show a system 100 for disinfecting pipes or conduits. The system 100 comprises a conduit 50 and an ozone generation apparatus 10. The conduit 50 has an inlet 52, an outlet 54, and an interior volume 56. The interior volume 56 is in fluid communication with the inlet 52 and outlet 54. The ozone generation apparatus 10 is in fluid communication with the inlet 52. In the embodiment shown in FIGS. 1A and 1B, the conduit 50 is substantially free of liquid. Substantially free of liquid can mean that liquid comprises less than 10% of the conduit 50 by volume, less than 5% of the conduit 50 by volume, or less than 1% of the conduit by volume. The system of FIGS. 1A and 1B may further comprise an ozone destruction apparatus 70 (shown in FIG. 2), which can be placed in fluid communication with the conduit outlet 54. The ozone apparatus 70 will be discussed in greater detail below.

Referring now to FIG. 2, a system 200 for disinfecting pipes or conduits is provided. The system 200 comprises a conduit 50, an ozone generation apparatus 10, and an ozone destruction apparatus 70. The conduit 50 has an inlet 52, an outlet 54, and an interior volume 56. The interior volume is in fluid communication with the inlet 52 and 54. The ozone generation apparatus 10 is in fluid communication with the inlet 52 and the ozone destruction apparatus 70 is in fluid communication with the outlet 54.

The conduit 50 in FIGS. 1, 2, and 3 can be comprised of many different materials, including but not limited to metals, plastics, cements, any other material suitable for transporting fluid, or combinations thereof. The conduit 50 may be comprised of steel, galvanized steel, galvanized iron, cast iron, ductile iron, copper, lead, bronze, or any number of alloyed materials. The conduit 50 may also be comprised of polyvinyl chloride (PVC), chlorinated polyvinyl chloride (CPVC), cross-linked polyethylene (PEX), polypropylene (PP), polybutylene terephthalate (PBT), acrylonitrile butadiene styrene (ABS) or other polymeric materials. The conduit 50 may also be comprised of composite materials, such as PEX-Al-PEX, which may include different layers of materials to harness the advantages of multiple materials. The conduit 50 may also be comprised of clay, tile, or other materials that may be used to transport fluid. Additionally, the conduit 50 may be lined with different materials, such as PVC, cement, or flexible plastics.

The conduit 50 has an inner surface 51 and an outer surface 53 that define the cross-sectional shape of the conduit 50, as seen in FIG. 1B. The conduit also has a thickness t that spans between the inner surface 51 and the outer surface 53. The outer surface 53 may be rounded, as depicted in FIGS. 1, 2, and 3, or may take any number of other shapes. The inner surface 51 may also be rounded, as depicted in FIGS. 1 and 2, or may take any number of other shapes, for instance triangular, oval, or square. In some embodiments, the inner surface 51 and outer surface 53 will be concentric with one another, forming an annular cross-section, as shown in FIG. 1B. In some non-limiting examples, the conduit 50 has a cylindrical shape.

The conduit 50 may have any range of lengths and diameters. For instance, the conduit outer surface 53 may have a diameter of between 1 inch and 168 inches. The conduit 50 may have any length between several feet and several thousand feet. For example, the conduit 50 may be about 1,200 feet long. The conduit thickness t may range anywhere between 0.02 inches and 100 inches. The thickness t may be uniform throughout the conduit 50, or may vary.

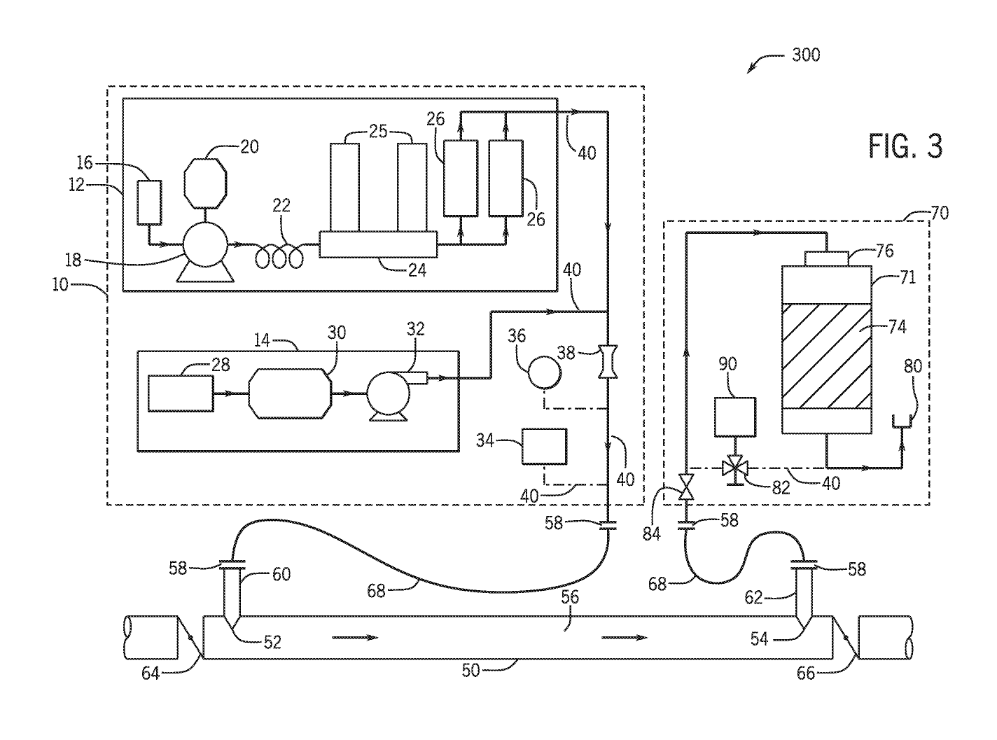

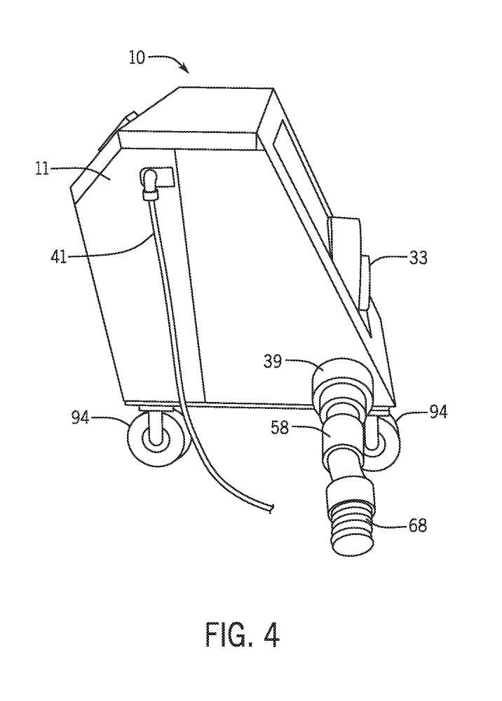

Referring now to FIG. 3, a schematic view of a non-limiting example of a system 300 for disinfecting pipes or conduits, similar to systems 100 and 200, is provided. The system 300 comprises an ozone generation apparatus 10, a conduit 50, and an ozone destruction apparatus 70, as shown and described in detail in FIGS. 4-8.

The ozone generation apparatus 10 may comprise an oxygen concentrator system 12 and an air blower system 14. The oxygen concentrator system 12 is used to concentrate oxygen in order to produce ozone, while the air blower system 14 is used to dilute ozone produced by the apparatus 10 and/or push fluid through the conduit inlet 52 through the conduit interior volume 56 and through the conduit outlet 54. It should be appreciated that the components described as belonging to certain systems like the oxygen concentrator system 12 or air blower system 14 can be present in the invention even in embodiments that omit these systems. For instance, an embodiment of the invention may not include an oxygen concentrator system 12, but may still include an air compressor 18.

The oxygen concentrator system 12 may comprise several components, as seen in FIG. 3. In some embodiments, the oxygen concentrator system 12 includes an air compressor 18. The air compressor may comprise a compressor motor 20, an air intake filter 16, and a cooling coil 22. Air can be drawn into the compressor 18 through the air intake filter 16, which may filter out solid debris. The motor 20 rotates, causing the compressor 18 to compress the air. The compressed air may then be passed by or through the cooling coil 22, which can cool down air coming out of the air compressor 18. The cooling coil 22 may be made of aluminum or copper, or other suitable metals that can perform cooling functions.

The air may then be passed to the oxygen concentrator 24, which may comprise one or more sieve beds 25. A sieve bed 25 comprises molecular sieve materials that can absorb nitrogen, carbon dioxide, and water vapor from the compressed air that enters the oxygen concentrator 24, so that a mixture comprising of a majority of oxygen and argon passes through to the ozone reaction chamber 26. Using an oxygen concentrator 24, atmospheric feed gas which is about 21% oxygen can be filtered such that the resulting gas has an oxygen level over 90%. Using concentrated oxygen has been shown to increase ozone production by a factor of approximately four.

Alternatively, in some embodiments, a compressed pure oxygen supply, a compressed air mixture, gaseous oxygen, or air tanks can be provided. If such an oxygen or air source is provided, the ozone generation apparatus 10 may or may not comprise a compressor 18. Similarly, ozone generation apparatuses 10 that make use of these alternative air supplies may or may not comprise oxygen concentrators 24 or a sieve bed 25.

The ozone generation apparatus 10 may comprise one or more ozone reaction chambers 26, which house the reaction that creates ozone. Ozone may be produced in a number of ways, for instance, by a corona discharge (CD) generator or ultraviolet generation. CD generators produce ozone gas by supplying a high voltage across an air gap. By subjecting the compressed oxygen and argon to a high voltage, a percentage of the oxygen flowing through the air gap is turned to ozone. To adjust the concentration of ozone produced in the one or more ozone reaction chambers 26, the ozone generation apparatus 10 may further comprise an electrical potentiometer (not shown) that can adjust the power delivered to the ozone generation apparatus 10. By varying the power delivered to the ozone generation apparatus 10, the voltage supplied across the air gap in the ozone reaction chamber 26 can be varied, thereby affecting the ozone production rate. By controlling the voltage across the air gap, certain desirable concentration rates of ozone can be obtained. In another embodiment of the invention, the outlet 54 of the conduit 50 can be restricted by a valve such as valve 84 which reduces air flow and increases the ozone concentration. Ozone concentrations may range from 0 to 75,000 ppm, or even higher. For example, a desirable ozone concentration, after dilution by an air blower, may be about 1,500 ppm. Ozone concentration rates may also be measured in other units. For instance, ozone production may occur at 170 grams per hour at 6.0% concentration by weight.

While a percentage of the feed gas provided to the ozone reaction chambers 26 may be turned into ozone, a percentage of the feed gas will remain in its prior state. The ozone and air from the blower 32 will combine to form a diluted ozone gas/air mixture, which can then be introduced into the conduit 50. As stated before, the ozone concentration coming out of the ozone generation apparatus 10 may vary between 0 to 75,000 ppm or higher, until the ozone output is combined with air from blower 32, which may then reduce the ozone concentration to be around 1,500 ppm.

Prior to entering the conduit 50, the ozone gas/air mixture can be passed through an ozone gas flow meter 38, as seen in FIG. 3. The ozone gas/air mixture flow meter 38 will read the flow rate of ozone gas/air entering conduit 50. In addition to the ozone gas/air mixture flow meter 38, the system may further comprise a gas pressure gauge 36 in fluid communication with the ozone gas/air mixture flow meter 38. Similarly, the system may further comprise an ozone analyzer 34 also in fluid communication with the ozone gas/air mixture flow meter, which may display the concentration of ozone in the ozone gas/air mixture which will be supplied to the conduit 50. The concentration of ozone in the ozone gas/air mixture can be adjusted manually or automatically. For example, ozone concentration can be controlled by adjusting power to the ozone generation apparatus 10, adjusting the shutoff valve 84 at the outlet of the conduit 54, or by otherwise adjusting the amount of air provided by the blower 32.

The compressed air and ozone gas may be transported through the oxygen concentrator system 12 through a variety of ways. For instance, tubing 40 may be used to transport the gases. The tubing 40 can comprise a number of ozone-resistant materials, including certain metals, plastics, and composites. For instance, the tubing 40 may comprise schedule 80 PVC pipe, 316 stainless steel or other ozone-resistant materials. The tubing may have a variety of cross sectional shapes, including annular or square. In some embodiments, tubing 40 comprises a 316 stainless steel braided hose.

As mentioned earlier, the ozone generation apparatus 10 may further comprise an air blower system 14, as shown in FIG. 3. The air blower system 14 may comprise a variable frequency drive 28, a blower motor 30, and an air blower 32. The variable frequency drive 28 allows the blower motor 30 to be sped up or slowed down, which affects the rate fluid is displaced by the blower system. The variable frequency drive 28 may be controlled manually, or may be controlled electronically. The blower motor 30 may be any known motor acceptable for use in a blower system. For example, the blower motor may be a 3 phase, 5 horsepower induction motor. The air blower 32 may comprise one or more blades, which rotate angularly to propel air. The air blower 32 may comprise a casing to direct the flow of air in a desired direction. The blower system 14 may optionally include an air intake and filter apparatus, which may filter the air prior to use in the blower system.

The air blower system 14 may serve a variety of purposes. In some embodiments, the air blower system 14 is used to purge the conduit of liquid before the disinfection process begins by blowing air into the system. The air blower system 14 may also be used to push a generated ozone/air mixture into the conduit inlet 52. In some non-limiting examples, the blower system 14 is used to push the ozone gas/air mixture out of the conduit interior volume 56 through to the conduit outlet 54 and into the ozone destruction apparatus 70. This can increase the velocity of the ozone through the pipeline, such that the travel time of the ozone gas from conduit inlet 52 to outlet 54 is reduced. By reducing the travel time of the ozone gas mixture, the total disinfecting cycle time of the pipeline is also decreased. In larger diameter conduits, this feature may decrease cycle time by several hours. Additionally, the air blower system 14 may dilute the ozone gas stream to provide a much larger volume of gas into the conduit 50. This dilution of the ozone mixture will reduce the concentration of ozone down to safer, but still effective levels, and can improve the travel time from conduit inlet 52 to conduit outlet 54. In higher pressure applications, it should be appreciated that an additional air compressor could be used to deliver air to the conduit 50. In some embodiments, the additional air compressor would take the place of an air blower system 14, and the air blower system 14 would be omitted from the ozone generation apparatus 10.

The ozone generation apparatus 10 may be powered in a variety of ways, including battery, a standard 110 VAC wall-mount, an independent generator, or other known power supplying mechanisms. For instance, the ozone generation apparatus 10 may run on 240 VAC, 20 Å power, supplied by an independent generator.

The ozone generation apparatus 10 can be a mobile trailer-mounted or cart-mounted device, or can be stationary. In some embodiments, the ozone generation apparatus 10 is equipped with one or more wheels 94, as shown in FIG. 4. The wheels 94 allow for easy transport of the ozone generation apparatus 10, and may promote wider use of the system 300. The wheels 94 may be comprised of several different materials, including inflatable rubber wheels or wheels made of a solid material, such as polyurethane. Additionally, the wheels 94 may comprise metals, such as steel or aluminum. The increased mobility of the system 300 enables the disinfection of remote pipeline areas in a water distribution system. The ozone generation apparatus 10 also does not require a pressurized water source to operate, unlike prior ozone solution systems and methods that required the use of a fire hydrant. This further adds to the mobility and versatility of the present system 300. In some embodiments, the ozone generation apparatus 10 is trailer-mounted, which may allow the apparatus to accommodate larger capacity ozone production, provide increased space for equipment storage and operation, provide space for a power supply generator and fuel tank (not shown), improve weather protection capabilities, or some combination of these features.

The system 300 for disinfecting pipes or conduits can be used to disinfect a conduit 50. In some non-limiting examples, the conduit 50 is first drained of liquids before ozone is introduced into the conduit 50. The parameters and the material selection of the conduit 50 can be similar to those described above. The conduit 50 may preferably be substantially free of liquid, like the conduit 50 shown in FIG. 1. Again, substantially free of liquid can mean that liquid comprises less than 10% of the conduit 50 by volume, less than 5% of the conduit 50 by volume, or less than 1% of the conduit 50 by volume.

The conduit 50 comprises an inlet 52, an outlet 54, and an interior volume 56, in fluid communication with the inlet and outlet. The interior volume of the conduit 56 may be the target area for disinfection. The ozone generation device 10 can be placed in fluid communication with the inlet 52. The line between the ozone generation device 10 and the inlet 52 may comprise pipes or hoses. For example, a hose 68 can be connected to hose fittings 58 that may be attached to the ozone generation apparatus 10 and a conduit inlet pipe 60. The hose fittings 58 may be of several different varieties, such as threaded, quick-connect, compression fit, or other types of fittings known in the art. The hose fittings 58 may be comprised of a number of ozone-resistant materials, including different plastics, metals, and composites. In some embodiments, stainless steel camlock hose fittings are used.

The conduit inlet pipe 60 may be a riser pipe in fluid communication with the conduit inlet 52. The conduit inlet pipe 60 may be comprised of a number of materials, similar to those materials that may comprise the conduit 50. The conduit inlet pipe 60 may be connected to the conduit inlet 52 through a corporation tap in the conduit 50. The corporation tap may be screwed into threads cut into the conduit 50, welded or brazed to the conduit 50, or attached in other means so as to create a water-tight seal between the conduit inlet pipe 60 and the conduit inlet 52. The conduit inlet pipe 60 may optionally comprise a shut-off valve that can prevent fluid from flowing in or out from the conduit. The conduit inlet pipe 60 may also comprise a hose fitting 58 as described above. In some embodiments of the invention, the conduit inlet pipe 60 and ozone generation apparatus 10 comprise camlock hose fittings, and are in fluid communication with a hose 68. The hose 68 can comprise several different ozone-resistant materials. For instance, a hose comprising stainless steel, PVC, or polytetrafluoroethylene may be used. In some embodiments, the hose 68 may be a 316 stainless steel braided hose.

The conduit 50 may also comprise isolation valves that prevent fluid from other conduits from entering the conduit 50. The system 300 can comprise an inlet isolation valve 64 and an outlet isolation valve 66. These valves 64, 66 may be butterfly valves, gate valves, ball valves, globe valves, diaphragm valves, or other valves known in the art. In some embodiments of the invention, the inlet isolation valve 64 and the outlet isolation valve 66 are the same type of valve. In other embodiments, different valves can be used. In some non-limiting examples, both the inlet isolation valve 64 and the outlet isolation valve 66 are closed when the disinfection process is occurring (e.g., the processes 500, 600 described below).

The conduit outlet 54 may also be in fluid communication with an ozone destruction apparatus 70. The line between the ozone destruction apparatus 70 and the outlet 54 may comprise pipes and hoses. For example, the hose 68 can be connected to hose fittings 58 that may be attached to the ozone destruction apparatus 70 and the conduit outlet pipe 62. The hose fittings 58 may be of several different varieties, such as threaded, quick-connect, compression fit, or other types of fittings known in the art. The hose fittings may be comprised of a number of ozone-resistant materials, including different plastics, metals, and composites. In some embodiments, stainless steel camlock hose fittings are used.

The conduit outlet pipe 62 may be a riser pipe in fluid communication with the conduit outlet 54. The conduit outlet pipe 62 may be comprised of a number of materials, similar to those materials that may comprise the conduit 50. The conduit outlet pipe 62 may be connected to the conduit outlet 54 through a corporation tap in the conduit 50. The corporation tap may be screwed into threads cut into the conduit 50, welded or brazed to the conduit 50, or attached in other means so as to create a water-tight seal between the conduit outlet pipe 62 and the conduit outlet 54. The conduit outlet pipe 62 may optionally comprise a shut-off valve that can prevent fluid from flowing in or out from the conduit. The conduit outlet pipe 62 may also comprise a hose fitting 58 as described above. In some embodiments of the invention, the conduit outlet pipe 62 and ozone destruction apparatus 70 comprise camlock hose fittings, and are in fluid communication with a hose 68.

The system 300 for disinfecting pipes or conduits can include an ozone destruction apparatus 70. The ozone destruction apparatus 70 is in fluid communication with the conduit outlet 54. The ozone destruction apparatus 70 may optionally comprise an ozone destruction vessel 71, an offgas outlet 80, and an ozone analyzer 90. Ozone may be converted back to oxygen gas in the ozone destruction vessel 71. The offgas outlet 80 may allow gas back into the atmosphere once it has been properly treated, or may allow gas back into the atmosphere without treatment. The ozone analyzer 90 may optionally monitor the concentration of ozone present in the conduit and other components which are in fluid communication with the conduit.

The ozone destruction vessel 71 may embodied as a mobile cart on wheels to allow for easy positioning at the conduit outlet 54. The ozone destruction vessel 71 may be comprised of several different materials, including plastics, metals, or composites. In some non-limiting examples, the ozone destruction vessel 71 comprises a stainless steel outer wall. The ozone destruction vessel 71 has an opening 76 where fluid from the conduit may flow in. Within the ozone destruction vessel 71 is a catalyst 74 which is capable of destroying ozone by converting it into oxygen. The catalyst may be comprised of manganese dioxide, ferric oxide, platinum, or combinations thereof, as well as other known catalysts for converting ozone to oxygen. In some embodiments of the invention, the catalyst is heated. The catalyst may be heated using an electric heating element, or by other known means of heating. In some embodiments, the ozone destruction vessel 71 includes a capped top opening 76 which can be readily removable, allowing access to the catalyst 74 for inspection and replacement, if needed. The capped top opening 76 may be threaded, flanged, or otherwise connected to allow for easy removal.

In some embodiments, the system 300 may further comprise an ozone analyzer 34 or 90. In some non-limiting examples, such as that shown in FIG. 3, ozone analyzers 34 and 90 are located on both the ozone generation apparatus 10 and the ozone destruction apparatus 70. In other embodiments, the system 300 may comprise only an ozone analyzer 34 on the ozone generation apparatus 10. In other non-limiting examples, the system may comprise only an ozone analyzer 90 on the ozone destruction apparatus 70. Other examples may not include an ozone analyzer 34, 90 at all. The ozone analyzer 90 may monitor the ozone gas concentration at the conduit outlet 54, or at other places throughout the conduit 50. The ozone analyzer 90 can be used to determine whether an adequate amount of ozone is present in the conduit 50, and can also be used to assure that all ozone is removed from the conduit 50 once the disinfection process is complete. The ozone analyzers 34 and 90 can be UV adsorption analyzers, electrochemical sensors, semiconductor based sensors (HMOS/GSS), or other types of ozone analyzers known in the art.

To enter the ozone destruction apparatus 70, fluid first passes through the conduit interior volume 56, the conduit outlet 54, and then may optionally pass through a shutoff valve 84, as seen in FIG. 3. The shutoff valve 84 can control whether or not fluid can enter the ozone destruction apparatus 70. For instance, when the conduit 50 is being disinfected, it may be advantageous to prevent an ozone gas/air mixture from entering the ozone destruction apparatus 70. When the shutoff valve 84 is closed, the ozone gas/air mixture may stay within the conduit 50 and continue the disinfection process. This may prove useful when the pipe is initially being filled with ozone gas/air mixture, so that the concentration of ozone can build.

In some embodiments, a pressure relief valve is supplied on the inlet of the ozone destruction apparatus 70. To maintain a minimum pressure in the conduit. This embodiment may also include a bypass valve to allow the pressure relief valve to be bypassed with filling or purging the system of ozone. A pressure relief valve, or other restricting valve such as valve 84 can be used to create back pressure inside the conduit 50. This restricting valve can also be used to increase or decrease the flow of the ozone gas/air mixture thus decreasing or increasing the ozone concentration in the ozone gas/air mixture.

When the isolation valves 64 and 66 are closed and the shutoff valve 84 is also closed, the conduit 50 can become pressurized. The conduit 50 may be pressurized anywhere from atmospheric pressure to 15.0 psi, or even greater. In some embodiments, the conduit 50 pressurizes to about 2-5 psi gauge pressure. The pressure in the conduit 50 will force ozone into every pore of the conduit inner surface 51, effectively disinfecting the entire conduit interior volume 56.

When the shutoff valve 84 is open, the ozone gas/air mixture may enter the ozone destruction apparatus 70. In some scenarios, like prior to introducing ozone into the conduit 50, it may be advantageous for fluid to bypass the ozone destruction vessel 71. To accommodate for these scenarios, the ozone destruction apparatus 70 may further comprise a three-way ozone gas valve 82. In one position, fluid would pass into the ozone destruction vessel opening 76 and the ozone analyzer 90, while in the other position, fluid would avoid the ozone analyzer and could pass through to the offgas outlet 80. In embodiments that do not have an ozone analyzer 90, the three-way ozone gas valve 82 could be replaced by a standard two-way valve, such as a butterfly valve, gate valve, globe valve, diaphragm valve, ball valve, or other valve known in the art. In another embodiment, valve 82 could be a three-way ozone gas valve allowing the ozone analyzer 90 to draw a sample from the pre-ozone destruct gas flow, or draw a sample from the post-ozone destruct gas flow to allow the operator to monitor both the concentration of the gas coming out of the conduit 50 as well as make sure the destruct is functioning properly.

In some non-limiting examples of the system 300, the ozone destruction apparatus 70 comprises inlet piping and a valve arrangement such that gas is diverted into the ozone destruction vessel 71 and liquid is directed to an external drain. This would prevent liquid from entering and possibly damaging any part of the ozone destruction vessel 71. In particular, it would prevent possible damage to the catalyst 74. This optional feature may prove useful if ozone gas/air mixture was being expelled from the conduit interior volume 56 by injecting liquid into the conduit inlet pipe 60 to displace the ozone gas/air mixture. The ozone gas/air mixture would still be directed into the ozone destruction vessel 71 to be converted back to oxygen and air, and the liquid, such as water, would exit out a drain without passing through the ozone destruction vessel 71. The feature is optional, as in some embodiments, ozone gas/air mixture is forced into the ozone destruction apparatus 70 by injecting compressed air or by forcing fluid through the conduit 50, such as through a process performed by the blower system 14.

Fluid may be transported through the ozone destruction apparatus 70 through a variety of ways. For instance, tubing 40 may be used to transport the gases. The tubing 40 can comprise a number of ozone-resistant materials, including metals, plastics, and composites. For instance, the tubing 40 may comprise PVC braided stainless steel, or other materials. The tubing 40 may have a variety of cross-sectional shapes, including annular or square.

The ozone destruction apparatus 70 may be powered in a variety of ways, including by battery, a standard 110 VAC wall-mount, an independent generator, or other known power supplying devices. For instance, the ozone destruction apparatus 70 may run on a DC battery.

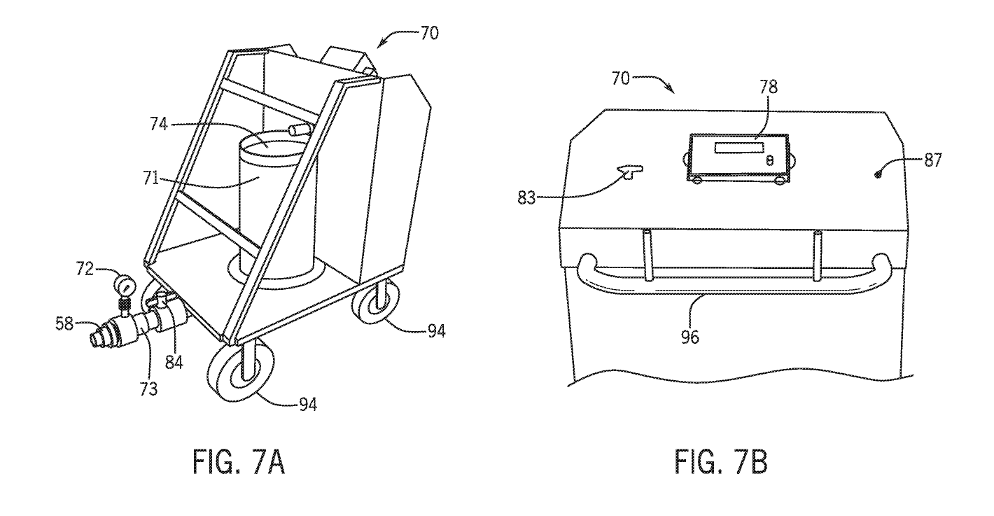

Additionally, the ozone destruction apparatus 70 may be a mobile cart-mounted or trailer-mounted device, or can be stationary. In some embodiments, the ozone destruction apparatus 70 is equipped with one or more wheels 94, as shown in FIG. 7A. The wheels 94 allow for easy transport of the ozone destruction apparatus 70, and may promote wider use of the system. The wheels 94 may be comprised of several different materials, including inflatable rubber wheels or wheels made of a solid material, such as polyurethane. Additionally, the wheels 94 may comprise metals, such as steel or aluminum. Similar to the ozone generation apparatus 10, the ozone destruction apparatus 70 may be trailer-mounted to allow the apparatus to accommodate larger capacity ozone destruction, provide increased volume for equipment storage and operation, improve weather protection capabilities, or some combination of these features.

In addition to the components shown in FIG. 3, it is further contemplated that all of the processes, such as adjusting pressure, switching valves, and controlling ozone production can be performed manually or automatically. Pneumatic valves, solenoid valves, or actuated valves capable of electronic control can be used in the system.

Controllers can be provided for the system, such that programs can be stored within the controller memory and later executed. For example, in systems comprising the ozone analyzer 34, the ozone analyzer 90, or both, the ozone analyzers 34, 90, the ozone generation apparatus 10, and a controller may be in electrical communication with one another. The ozone analyzers 34, 90, ozone generation apparatus 10, and controller may optionally be in electrical communication with the ozone destruction apparatus 70 as well. The controller can be configured to execute a program stored in the controller to adjust the rate of ozone production after a specified concentration of ozone is reached within the interior volume of the conduit 56, or when an ozone exposure time is reached within the interior volume 56, or when a calculated product of concentration of ozone and ozone exposure time is reached within the interior volume 56. In some embodiments, the controller may be prompted to execute a program based upon initial ozone concentration detected by ozone analyzer 34, final concentration of ozone gas detected by ozone analyzer 90, or a combination of both signals. In some examples, when the calculated product of ozone gas concentration “C” and ozone exposure time “T” exceed a certain amount, the controller can communicate to the ozone generation apparatus 10 that the disinfection cycle is complete, and ozone production can be stopped. In other non-limiting examples, when the ozone analyzer 34 or 90 detects ozone concentrations that are under desired values, the controller can communicate to the ozone generation apparatus 10 that the rate of ozone production must either be maintained or increase. In embodiments that produce ozone through corona discharge generators, the signal can prompt the voltage across the air gap to be varied, which would adjust the rate of ozone production to desired levels. Alternatively, the ozone gas/air mixture flow can be altered by a valve in the system like valve 84 on the outlet of the conduit which can increase or decrease the ozone concentration in the ozone gas/air mixture.

Similarly, the controllers may have stored time values that can prompt the opening and closing of various valves in the system. Timers may be provided to the system such that the controller can prompt system changes when a certain time value is observed. For instance, when a specified time is reached, a controller can prompt the three-way ozone gas valve 82 to switch positions. Additionally, controllers may be used to control the blower system 14. For example, certain observed values (e.g., values above or below a threshold value deemed acceptable) may prompt system 300 changes, or a combination of timers and observed values prompt changes such that portions of the system 300 can be automated. For example, if a controller determined that the calculated product of ozone gas concentration “C” and ozone exposure time “T” exceeded the required amount, the controller could communicate to the ozone generation apparatus 10 to cease ozone production and activate the blower system 14 to adjust the blowing rate. In some embodiments, the controller would communicate with the variable frequency drive 28.

It should be understood that the following Figures are in no way limiting or intended to define the proper or preferred orientation of components on any of the systems previously disclosed. Systems that include additional components or exclude some of the previously mentioned components are also within the scope of the invention, and the following Figures are provided only to provide an example of some embodiments of the invention.

FIG. 4 is a side view of an ozone generation apparatus 10 that can be used in systems 100, 200, and 300. The body 11 of the ozone generation apparatus 10 is comprised of a corrosion resistant material, for instance stainless steel or aluminum. Other components may protrude from the body 11 of the ozone generation apparatus 10. The blower vent 33, for example, can be seen on the exterior of the body 11 in FIG. 4. Wheels 94 can be seen below the body 11. In this embodiment, the ozone generation apparatus 10 comprises four wheels 94, each capable of full 360 degree rotation, such that the ozone generation apparatus 10 can be readily maneuvered. However, in some embodiments, one or more wheels 94 may be aligned rigidly, while other wheels maintain the ability to swivel. Additionally, a power cord 41 may be protruding from the body 11. In cases where the ozone generation apparatus 10 is battery powered, this part could be optional. The power cord may have a standard 110 VAC outlet plug on the end, a 240 VAC outlet plug, or any other known plug that connects the ozone generation apparatus 10 to a power source.

The ozone generation apparatus outlet 39 is shown protruding from the body 11 as well. The ozone generation apparatus outlet 39 is in fluid communication with a hose fitting 58, as described earlier. As shown in FIG. 4, a hose 68 is connected to the hose fitting 58. Ozone gas/air mixture, ambient air, or any other fluid can be expelled from the ozone generation apparatus 10 into the conduit through the ozone generation apparatus outlet 39.

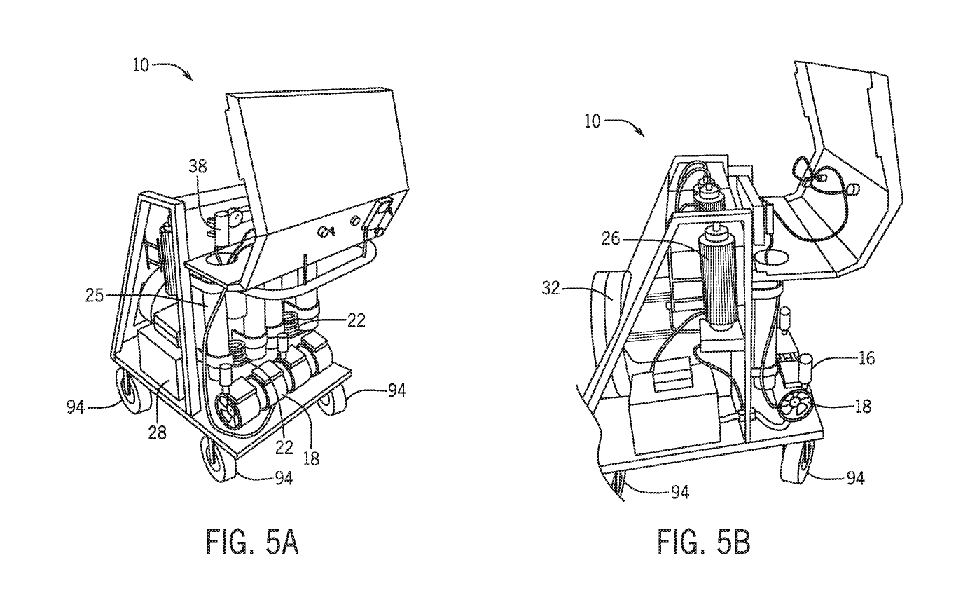

FIG. 5A shows the arrangement of internal components on the ozone generation apparatus 10. The ozone gas flow meter 38 can be seen near the top of the cart. Considering that it may be desirable to read these values, an ozone generation apparatus 10 may optionally have a digital display that is in electrical communication with the ozone gas flow meter 38. The compressor 18 is visible near the bottom of the ozone generation apparatus 10, as are the cooling coils 22. The variable frequency drive 28 is also located near the bottom of the ozone generation apparatus 10, opposite from the compressor 18. In the illustrative embodiment, the sieve bed 25 is housed in a vertically positioned cylindrical housing near the compressor 18.

With further reference to FIG. 5B, additional internal components of the ozone generation apparatus 10 are shown in their respective positions on the ozone generation apparatus 10. The air blower 32 is positioned such that it can draw air from the atmosphere. The orientation of the air blower 32 can be in any number of directions. The compressor air filter 16 can be positioned on top of the compressor 18. Several ozone reaction chambers 26 can be present within the ozone generation apparatus 10 as well.

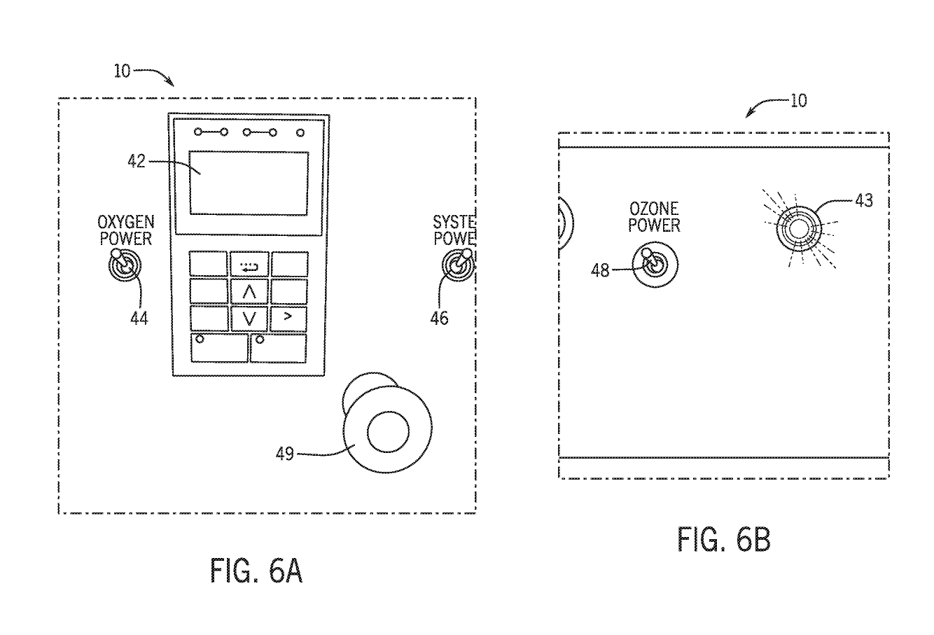

FIG. 6A shows a control panel that can be used on an ozone generation apparatus 10 according to embodiments of the disclosure. The control panel comprises a digital operator 42, an oxygen power toggle switch 44, a system power toggle switch 46, and an emergency stop button 49. The digital operator 42 may provide a variety of different information to a user, such as readings from pressure gauges, voltage supplied in the ozone reaction chamber 26, or ozone concentration data. Additionally, the digital operator 42 may provide time values, calculated concentration requirements, and may take user input to calculate system output, such as the dimensions of the conduit 50 to be disinfected. The digital operator 42 can allow a user to monitor the entire system 100, 200, 300 in some embodiments, and can actuate valves or adjust parameters of the system 100, 200, 300. The oxygen power toggle switch 44 may control the power to the oxygen concentrator system 12. The system power toggle switch 46 may control whether or not the entire ozone generation apparatus 10 is powered. The emergency stop button 49 may cause complete system shutdown when pushed.

FIG. 6B shows additional components that may be included on a control panel for an ozone generation apparatus 10. The control panel may further comprise an ozone power toggle switch 48 that controls when ozone is being produced. In some embodiments, when the ozone power toggle switch 48 is switched “off,” no voltage is supplied across the gap within the ozone reaction chamber 26, such that no ozone will be produced by the ozone generation apparatus 10. Finally, the control panel may comprise a power indicator 43 that can display any number of things. It can flash when ozone power is on, when system power is on, when the oxygen system is on, or for any number of other reasons.

The control panel may further comprise several other displays and switches as well. For instance, in an automated system it may be desirable to have a touch screen (not shown) to enter data or to quickly navigate and observe the status of various components of the system. In some embodiments, the control panel can provide voice instructions to a user with instructions on how to manually adjust certain features if the system requires some adjustment. Additionally, the system may further comprise an indication system that provides an alert when the system needs maintenance.

FIG. 7A shows a non-limiting example of an ozone destruction apparatus 70 that can be used in systems 100, 200, and 300. Ozone gas/air mixture enters the apparatus through the ozone destruction apparatus inlet 73. The ozone destruction apparatus inlet 73 is in fluid communication with a hose fitting 58, as described previously. Although not pictured, the hose fitting 58 would optionally be in fluid communication with a hose 68 during operation. The pressure of the gas is monitored by the ozone destruction apparatus inlet gauge 72. If the shutoff valve 84 is open, gas will enter into the ozone destruction vessel 71. In the pictured embodiment, a catalyst 74 is present in the ozone destruction vessel 71. The ozone gas/air mixture would react with the catalyst, such that ozone would be converted into oxygen gas. After conversion, the gases are released back into the atmosphere. In this embodiment, the ozone destruction apparatus 70 also comprises wheels 94.

FIG. 7B shows a non-limiting example of a control panel that can be used on an ozone destruction device 70 in accordance with embodiments of the disclosure. The control panel pictured comprises an ozone gas concentration monitor 78, a main line valve control 83, and a system power toggle switch 87. The ozone gas concentration monitor 78 relays the concentration of ozone gas in the ozone gas/air mixture entering the ozone destruction apparatus 70. In some embodiments, the ozone gas concentration monitor 78 is in electrical communication with an ozone analyzer 90 that reports the concentration of ozone. The main line valve control 83 pictured is a manual switch that can operate the shutoff valve 84, such that gas can be allowed into the ozone destruction apparatus 70. Additionally, it may control other valves in the system. In some embodiments, the valve controls are automated, such that the main line valve control 83 would be omitted. The system power toggle switch 87 of the ozone destruction apparatus 70 can be used to control whether or not the system is powered. Finally, the ozone destruction apparatus 70 may also comprise a push handle 96 that allows for easier pushing of the ozone destruction apparatus 70.

The control panel may further comprise several other displays and switches as well. For instance, in an automated system it may be desirable to have a touch screen to enter data or to quickly navigate and observe the status of various components of the system. In some embodiments, the control panel can provide voice instructions to a user with instructions on how to manually adjust certain features if the system requires some adjustment. Additionally, the system may further comprise an indication system that provides an alert when the system needs maintenance.

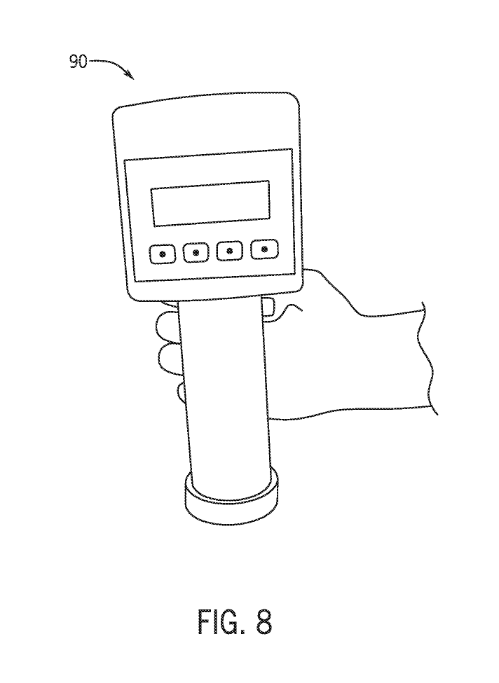

FIG. 8 shows an example of an ozone analyzer 34 or 90 that can be used in the system 100, 200, or 300. Although other types of ozone analyzer 34, 90 can be used as previously discussed, the portable ozone sensor shown in FIG. 8 (which is available from Analytical Technology, Inc.) is a reliable UV adsorption analyzer that can perform well in systems 100, 200, and 300. This optional device can be connected to a system 100, 200, or 300 in a number of ways, and can be physically connected to the system, electrically connected to the system, or both.

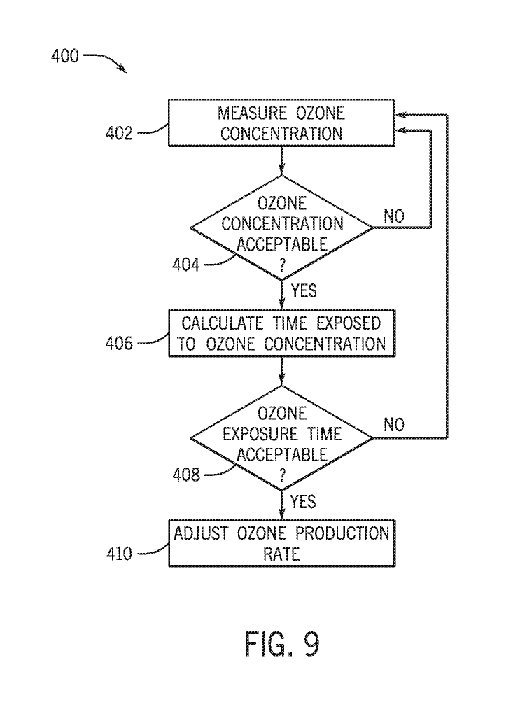

FIG. 9 shows a block diagram of a program 400 that could be performed by a controller in an ozone gas disinfection system such as the systems 100, 200, or 300 previously discussed. The program 400 could be executed on a controller in a system that comprises an ozone analyzer 34, 90. At block 402, the ozone concentration within the conduit 50 is measured by the ozone analyzer 90. The measured value of ozone concentration is compared with desired ozone concentration values at block 404. If the concentration is not acceptable, the controller may optionally communicate to the ozone generation apparatus 10 to adjust the ozone production rate. Alternatively, the valve on the outlet of the conduit valve 84 can be adjusted manually or automatically until the desired ozone concentration is achieved. Then the process 400 will return to measuring the ozone concentration in the conduit at block 402. If the measured ozone concentration is determined to be acceptable at block 404, the controller may then begin to start a timer at block 406. The ozone analyzer 90 will continuously monitor the ozone concentration within the conduit 50, and as long as the ozone concentration stays above a desired level, the ozone generation apparatus 10 may not require adjustment.

Because disinfection requirements are measured in a concentration of the disinfectant multiplied by time exposed to a concentration of the disinfectant, the controller can store continuous data points of the concentration and times associated with them in the system at block 406. At block 408, the controller determines whether the product of the disinfectant concentration and exposure time has met or exceeded the sanitation requirements. If the product of concentration and time have not met or exceeded the sanitation requirements, the controller returns to measuring ozone concentration 402 and exposure time, until the observed value meets the sanitation standard.

If the product of ozone concentration and exposure time have met or exceeded the sanitation requirements, the controller can communicate to the ozone generation apparatus 10 that ozone production may be adjusted. For instance, it may be desirable to then turn the ozone concentration system 12 off when such values is observed. In addition to adjusting the rate of ozone production, the controller may communicate to the ozone generation apparatus 10 to adjust the air blower system 14. For instance, when the sanitation standard is met, the air blower system 14 can be prompted to increase blowing rate, such that the ozone gas/air mixture within the conduit 50 is blown out of the system and into the ozone destruction apparatus 70. Additionally, the controller may communicate to the ozone destruction apparatus 70 to open the shutoff valve 84, so as to allow gas to flow into the ozone destruction apparatus 70 from the conduit outlet 54.

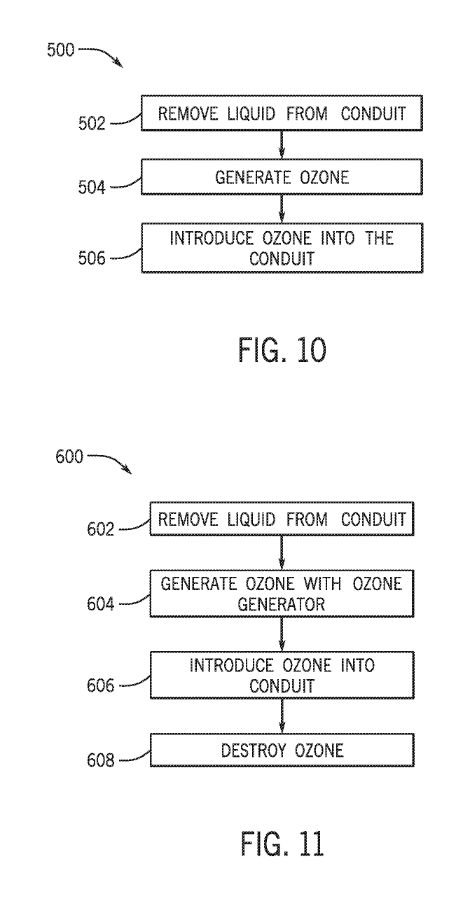

A process for disinfecting conduits or pipes is shown in FIG. 10. The process 500 can be performed using any of the systems 100, 200, 300 discussed previously, and is described as being performed by various components within the system 300. The process 500 involves first removing liquid from a conduit, such as the conduit 50, having an inlet 52, outlet 54, and an interior volume 56 in fluid communication with the inlet 52 and the outlet 54, at block 502. The amount of liquid removal may vary. In some embodiments of the process 500, liquid may be removed until the conduit 50 is substantially free of liquid. Substantially free of liquid can mean that liquid comprises less than 10% of the conduit by volume, less than 5% of the conduit by volume, or less than 1% of the conduit by volume. In some embodiments, prior to removing fluid from the conduit 50, the conduit 50 is first isolated from other conduits. This step may occur by closing two isolation valves 64, 66 coupled to the conduit 50.

The liquid can be removed from the conduit 50 in a variety of ways. For instance, liquid can be drained from the conduit 50 through a drain opening in the conduit. Additionally, liquid can be blown out of the conduit 50. In some embodiments, the conduit 50 is purged of liquid by forcing compressed air through the conduit 50. In other embodiments, the conduit 50 is purged of liquid by a blower system 14, as previously described. The liquid removal process 502 can be aided by an ozone generation apparatus 10, or can be performed through other known methods of fluid removal. In some embodiments, a plastic pigging device called a “pig” may be used with compressed air and the blower system 14 to push the pig through the pipe. This can push out any water filled areas at low points in the conduit 50.

Once the step 502 of removing liquid has been performed, an ozone generation apparatus 10 generates gaseous ozone at block 504. The ozone generation apparatus 10 may generate ozone through a number of methods, including the CD method or ultraviolet generation. The ozone generation apparatus 10 may comprise any number of the features as described above for the ozone generation apparatuses of systems 100, 200, or 300.

Once the ozone is generated at block 504, the ozone is introduced into the interior volume 56 of the conduit 50 at block 506. The ozone can be introduced into the conduit 50 in a number of ways. In some non-limiting examples, this step 506 is performed by the blower system 14 described above. The blower system 14 dilutes the generated ozone gas and pushes the gas mixture into the conduit inlet 52 and into the interior volume 56 of the conduit 50. In some non-limiting examples, the generated ozone gas is delivered into the conduit inlet 52 with an air compressor 18. In other embodiments, the generated ozone is combined with compressed air and the pressure differential between the compressed air source and the conduit 50 causes the ozone gas/air mixture to spread into the interior volume 56 of the conduit 50.

In some non-limiting examples, when the ozone disinfection cycle is completed, a portion of the generated ozone is removed from the interior volume of the conduit. This step can be performed in a variety of ways, such as by blowing out the gas with the blower system 14. Alternatively, this step can be performed by introducing compressed air into the conduit inlet 52, and opening a valve 66 such that gas can escape out of the conduit outlet 54. This process can also be performed by introducing liquid, such as water, to flush the system and push out ozone gas that may be present in the conduit 50. In some embodiments, a fire hydrant may be put in fluid communication with the conduit 50 to perform this step.

In some embodiments, the process further comprises destroying a portion of the ozone. The ozone destruction process can occur in a number of ways. In some embodiments, the ozone destruction process can occur by bringing ozone gas into contact with a catalyst, such as manganese dioxide, ferric oxide, platinum, or combinations thereof, for example. In some embodiments, this step further comprises heating the catalyst.

Alternatively, the step of destroying a portion of the generated ozone may occur by pushing the ozone gas into an ozone destruction apparatus, similar to ozone apparatus 70 described above. The ozone destruction apparatus 70 may also utilize a catalyst, such as those mentioned above. When the ozone gas contacts the catalyst, it is converted back to oxygen.

In another embodiment of the disclosure, a process 600 for disinfecting conduits or pipes is shown in FIG. 11. In this embodiment, the process 600 begins by removing liquid from a conduit 50 at 602. The conduit 50 has an inlet 52, outlet 54, and an interior volume 56 in fluid communication with the inlet 52 and the outlet 54. The processes for removing liquid are similar to those processes disclosed in step 502 of process 500 for removing liquid from a conduit 50, and can be performed by a blower system 14 that blows liquid out of the conduit with forced air. In some embodiments, liquid in the conduit 50 can be drained from the conduit 50.

Next, ozone is generated by an ozone generation apparatus at block 604. Once again, the ozone may be generated by ozone generation apparatus 10 described above, or through other methods. In some embodiments, ozone is produced through a CD or ultraviolet generation method. Once the ozone is generated at block 604, the generated ozone is introduced into the interior volume 56 of the conduit 50 at block 606. Again, this step can be performed as was described in step 506 of process 500, by using pumping, blowing, or pressure differentials within the conduit 50 to drive ozone into the interior volume 56 of the conduit 50.

Finally, a portion of the generated ozone is destroyed at block 608. The destruction processes can occur in a number of ways, such as bringing the ozone into contact with a catalyst. The catalyst may be comprised of manganese dioxide, ferric oxide, platinum, or combinations thereof. Additionally, the step of destroying a portion of the generated ozone gas 608 may involve introducing the ozone gas into an ozone destruction apparatus, such as ozone destruction apparatus 70 discussed in detail above.

Embodiments of the process 600 may also comprise the step of isolating a section of a conduit 50. In some embodiments, this step will occur prior to the step 602 of removing liquid from the conduit. Isolating the conduit may occur by providing isolation valves 64, 66 on two ends of the conduit 50. By closing these isolation valves 64, 66, additional liquid from outside sources can be blocked from entering the conduit 50. Once the conduit 50 has been isolated, the process of removing liquid 602 can occur.

In some embodiments of the disclosure, once liquid has been removed from the conduit at step 602, the conduit 50 is purged of liquid using compressed air or blown air. This further removes liquid from the conduit 50, and may remove standing water that is left in the conduit 50. The process may further comprise the step of compressing air. The step of compressing air may be performed by a compressor 18 on an ozone generation apparatus 10, or may be performed by an independent compressor. The compressor 18 may take in ambient air through a filter prior to compressing the air.

In some non-limiting examples of the process 600, the process 600 further comprises the step of concentrating oxygen. In some embodiments, oxygen is concentrated by passing oxygen through one or more sieve beds 25, which filter air such that the resultant gas comprises a majority of oxygen and argon, with oxygen comprising a larger percentage of the mixture than in ambient air. When the air comes into contact with a sieve bed 25, much of the nitrogen, carbon dioxide, and water vapor can be removed. For instance, oxygen comprises about 21% of the makeup of ambient air, but after concentrating the oxygen, oxygen may comprise 90% or higher of the mixture.

The rates of ozone production may vary during the step of generating ozone 604. For instance, in some non-limiting examples of the process 600, the ozone may be produced between 0 ppm and 5000 ppm, or even higher. For example, a desirable ozone production concentration may be 1,500 ppm. Ozone concentration rates may also be measured in other units. For instance, ozone production may occur at 170 grams per hour at 6.0% concentration by weight.

Some embodiments of the process 600 further comprise holding the generated ozone in the conduit interior volume 56 for a period of time. To disinfect the conduit 50, the conduit 50 may need to be subjected to certain concentrations of ozone for a period of time to be adequately disinfected. Hold times may vary from one second to 24 hours, and even longer, depending on the size and parameters of the conduit 50 to be disinfected.

Any of the disclosed processes 500, 600 may also include the step of pressurizing the interior volume of the conduit to between atmospheric pressure and about 15 psi. The step of pressurizing the pipe may be performed by blocking potential escape routes for the ozone gas. In some embodiments of the method 500, 600, an exit valve can be closed. As more ozone gas and compressed air are introduced into the conduit interior volume 56, the conduit 50 pressurizes. While in some embodiments this pressure is between about 3-5 psi gauge, pressures both above and below this value can occur. In some embodiments, the pressure within the conduit 50 may be cycled. The pressure can be cycled by closing off the valve 84 and pressurizing the conduit interior volume 56 for a period of time, then opening the valve 84 and allowing the ozone concentration within the conduit interior volume 56 to increase (by releasing pressurized air from the conduit interior volume 56), then closing off the valve 84 and re-pressurizing the system. This cycle can be repeated many times.

In addition to the steps disclosed, in certain embodiments of the present disclosure, the processes 500, 600 further comprise monitoring the ozone concentration within the interior volume 56 of the conduit 50. This process can be performed using an ozone analyzer, such as ozone analyzer 34, 90 described above. The concentration of ozone may be monitored for the entire disinfection process, for part of the disinfection process, or may be omitted completely.

In certain embodiments of the disclosure, the process 500, 600 further comprises monitoring the time and ozone concentration simultaneously, which can be performed by a controller or a processor. Because CT is a desirable parameter to know, a controller may be configured to record data on both parameters. Using this data, the process may further comprise comparing the calculated CT values against acceptable CT values with the controller that may signal when the conduit 50 has been properly sanitized.

Once it has been determined that the conduit 50 has been exposed to the appropriate amount of ozone gas for the appropriate amount of time, the process may further comprise opening a valve to allow ozone gas to escape from the conduit interior volume 56. This valve may optionally be connected to an ozone destruction apparatus, such as ozone destruction apparatus 70 discussed earlier. Opening the valve may release some of the pressure present in the conduit interior volume 56, and may cause some of the ozone to exit the conduit interior volume 56 out the outlet 54.

In some non-limiting examples, the process 500, 600 further comprises flushing ozone gas from the conduit 50. In some embodiments, this process occurs by blowing air into the conduit 50 using the blower system 14. In other examples, the flushing process is carried out by introducing compressed air into the conduit 50, such that the compressed air pushes the ozone out of the conduit 50. In yet other non-limiting examples, the flushing process occurs by providing liquid to the conduit 50, such that all the gas is forced out. The liquid may be provided by a fire hydrant, or could be pumped into the conduit 50 by other means. In processes utilizing the flushing technique, the process 500, 600 may further comprise diverting gas flows to a catalyst and diverting liquid flows to an external drain, so as to prevent damage to the catalyst.

All processes described may further include releasing the oxygen gas back into the atmosphere. In some embodiments, the ozone is converted into oxygen gas when it contacts a catalyst. Once the ozone has been converted back into oxygen, it can be released into the atmosphere. In other embodiments, the process comprises allowing ozone to naturally decay back into oxygen gas before releasing it into the atmosphere.

All processes described may also be automated or partially automated, such that controllers can trigger certain events to occur, such as increasing blower system speed or increasing ozone concentration production. Valves may be actuated as well, and can be subject to automation.

### EXAMPLES

The following Examples have been presented in order to further illustrate the invention and are not intended to limit the invention in any way.

### Example 1

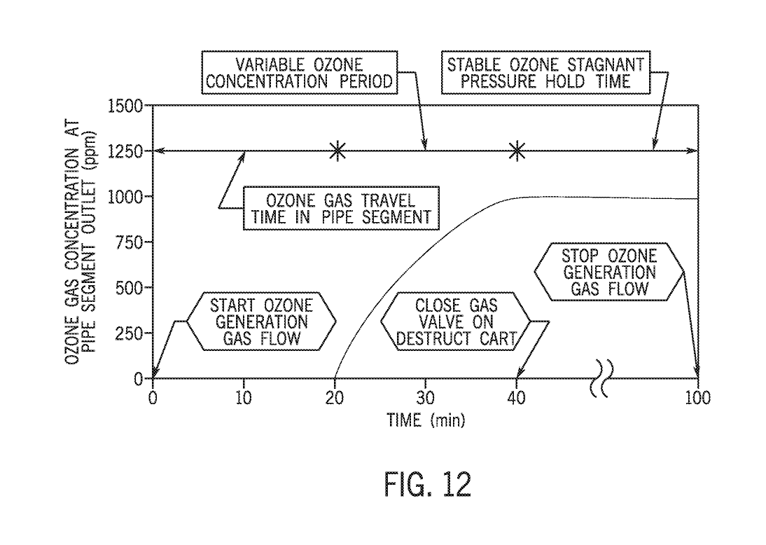

The timeline of FIG. 12 provides an example of a cycle time using the above disclosed process. At time t=0, the ozone generation process 500, 600 is started. Ozone is introduced into the conduit 50, and begins to travel throughout the conduit 50. The ozone concentration within the ozone generation apparatus 10 is monitored using the ozone analyzer 34. Using a Pressure Relief Valve or other type of valve 84, flow is limited until the ozone centration in the ozone gas/air mixture is at the desired level. At time t=20 minutes, ozone gas has reached the outlet 54 of the conduit 50, where the concentration of ozone is being measured. As ozone continues to fill the conduit 50, the measured ozone concentration continues to rise. At t=40 minutes, the ozone concentration hits a desired value, such as 1,000 ppm, for example. When the ozone concentration hits this value, the shutoff valve 84 on the ozone destruction apparatus 70 is closed, preventing gas from escaping the conduit 50. Between t=40 minutes and t=100 minutes, ozone within the conduit 50 is held at a stagnant pressure. At t=100 minutes, a desired CT value has been met. The ozone generation apparatus 10 can be turned off and the shutoff valve 84 on the ozone destruction apparatus 70 can be opened, such that ozone gas can be released from the conduit.

### Example 2

The benefits of the aforementioned systems and processes have been proven through lab and field testing. The inventive systems and processes have proven to be more effective than other widely used methods of disinfecting conduits used widely in the art.

Using embodiments of the aforementioned processes and methods, lab tests were performed on conduits of various diameters and lengths. To test various parameters of the disclosure, three different water mains were tested. These water mains included a 6-inch PVC pipe, a 6-inch ductile iron pipe, and an 8-inch PVC pipe. Along with the varying materials and sizes, different bacteria were placed into the water mains. The concentration of these bacteria was measured before disinfection, immediately after disinfection, and one week after the disinfection process. The exposure times were also varied between about 15 to about 60 minutes, although exposure times outside that range can be effective as well. Using embodiments of the inventive methods and systems, providing ozone gas to a conduit at 200 ppm proved effective at killing numerous types of different bacteria, as shown in the Table 1 below.

TABLE 1

| | | | | | Post- |
|---|---|---|---|---|---|
| Pipe Rack | | | Pre- | Post- | Disinfection |
| Segment | | | Disinfection | Disinfection | with 1-Week |
| No. | Disinfection Method | Test Organism | (MPN/100 mL) | (MPN/100/mL) | Hold (MPN/100 mL) |
| 1 | Ozone Gas 200 ppm | Pseudomonas | >2419 | <1 | <1 |
| 1 | Ozone Gas 200 ppm | T. Coli | 1 | <1 | <1 |
| 1 | Ozone Gas 200 ppm | E. Coli | <1 | <1 | <1 |
| 2 | Ozone Gas 200 ppm | Pseudomonas | >2419 | <1 | <1 |
| 2 | Ozone Gas 200 ppm | T. Coli | 2 | <1 | <1 |
| 2 | Ozone Gas 200 ppm | E. Coli | <1 | <1 | <1 |
| 3 | Ozone Gas 200 ppm | Pseudomonas | >2419 | <1 | <1 |
| 3 | Ozone Gas 200 ppm | T. Coli | <1 | <1 | <1 |
| 3 | Ozone Gas 200 ppm | E. Coli | <1 | <1 | <1 |

Thus, the invention provides improved systems and methods for disinfecting pipes or conduits. The invention provides improved process times, decreases wastewater, and produces superior disinfection.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A system for disinfecting pipes, the system comprising:
a conduit, the conduit having an inlet, an outlet, and an interior volume in fluid communication with the inlet and outlet; and
an ozone generation apparatus for producing gaseous ozone, wherein the ozone generation apparatus is in fluid communication with the inlet and the interior volume of the conduit is substantially free of liquid.

2. The system of claim 1 wherein the system is a mobile cart-mounted system.

3. The system of claim 1 wherein the system is a mobile trailer-mounted system.

4. The system of claim 1 further comprising riser pipes at the inlet and the outlet to introduce ozone and collect ozone offgas.

5. The system of claim 1 further comprising an ozone destruction apparatus, wherein the ozone destruction apparatus is in fluid communication with the outlet.

6. The system of claim 5, wherein the ozone destruction apparatus further comprises a liquid bypass system.

7. The system of claim 5, wherein a relief valve is positioned between the outlet and the ozone destruction apparatus, the relief valve configured to selectively restrict fluid flow from the outlet into the ozone destruction apparatus.

8. The system of claim 1, wherein the ozone generation apparatus further comprises a blower for moving fluid from the interior volume to the outlet.

9. The system of claim 1, wherein the ozone generation apparatus further comprises an ozone analyzer.

10. A system for disinfecting pipes, the system comprising:
a conduit, the conduit having an inlet, an outlet, and an interior volume in fluid communication with the inlet and outlet;
an ozone generation apparatus, wherein the ozone generation apparatus is in fluid communication with the inlet; and
an ozone destruction apparatus, wherein the ozone destruction apparatus is in fluid communication with the outlet.

11. The system of claim 10, wherein the system further comprises at least one ozone analyzer.

12. The system of claim 11, wherein the ozone analyzer is located on at least one of the ozone generation apparatus, ozone destruction apparatus, or both.

13. The system of claim 11, wherein the system comprises a controller in electrical communication with the ozone generation apparatus and the ozone analyzer, the controller being configured to execute a program stored in the controller to adjust the rate of ozone production after a specified concentration of ozone is reached within the interior volume, or an ozone exposure time is reached within the interior volume, or a calculated product of concentration of ozone and ozone exposure time is reached within the interior volume.

14. The system of claim 10, wherein the ozone destruction apparatus comprises a catalyst selected from the group consisting of manganese dioxide, ferric oxide, and platinum.

15. A process for disinfecting pipes, the process comprising:
removing liquid from a conduit, the conduit having an inlet, an outlet, and an interior volume in fluid communication with the inlet and the outlet;
generating ozone with an ozone generation apparatus; and
introducing the ozone into the interior volume of the conduit.

16. The process of claim 15, the process further comprising destroying a portion of the generated ozone.

17. The process of claim 15, the process further comprising blowing a portion of the generated ozone out of the interior volume.

18. The process of claim 15, the process further comprising introducing compressed air into the interior volume.

19. The process of claim 15, the process further comprising pressurizing the interior volume of the conduit to between 0.1 and 15 psi gauge pressure.

20. The process of claim 19, wherein the gauge pressure within the interior volume of the conduit is cycled by opening a relief valve downstream from the outlet to release fluid contained within the interior volume through the outlet, closing the relief valve, and introducing additional ozone into the interior volume of the conduit.
